(19) United States
(12) Patent Application Publication
(10) Pub. No.: US 2026/0263049 A1
(43) Pub. Date: Sep. 10, 2026

(54) ARTIFICIAL INTELLIGENCE-CONTROLLED ULTRASOUND SETUP FOR POWER AND IMAGE QUALITY OPTIMIZATION

(71) Applicant: GE Precision Healthcare LLC, Waukesha, WI (US)

(72) Inventors: Ole Blytt, Tonsberg (NO); Robert Schittny, Oslo (NO)

(73) Assignee: GE Precision Healthcare LLC, Waukesha, WI (US)

(21) Appl. No.: 19/071,529

(22) Filed: Mar. 5, 2025

(51) Int. Cl. A61B 8/00; G06T 7/00

(52) U.S. Cl. CPC A61B 8/5215; A61B 8/429; A61B 8/546; G06T 7/0002; G06T 2207/30168

(57) ABSTRACT

An ultrasound imaging system includes a transducer configured to transmit and receive an ultrasound signal, a matching layer, a damping block, and a processing circuit comprising a processor coupled to a memory device storing instructions thereon that, when executed, cause the processing circuit to perform operations including: determining, by a machine learning model, that the transducer is being navigated, dynamically adjusting, by the machine learning model, one or more acquisition parameters of the transducer to reduce an image quality of an image corresponding to the ultrasound signal, determining, by the machine learning model, that the transducer is positioned to capture a first ultrasound image, and dynamically readjusting, by the machine learning model, the one or more acquisition parameters to increase an image quality of the image corresponding to the ultrasound signal when the transducer is positioned to capture the first ultrasound image.

### FIELD

Embodiments of the subject matter disclosed herein relate to ultrasound imaging, and more particularly, to optimizing ultrasound image quality using artificial intelligence.

### BACKGROUND

During a medical imaging scan, a plurality of medical images of a patient are obtained by a technician, such as a sonographer, to measure or detect various aspects of anatomical features present within the medical images. Acquisition parameters used to obtain the medical images (e.g., a frequency, acquisition angle, dynamic power, gain, compound imaging, etc.) impact the resulting image quality of the medical images and may be adjusted to increase battery life of a wireless ultrasound probe.

### SUMMARY

An embodiment relates to an ultrasound imaging system including: a transducer configured to transmit and receive an ultrasound signal, a matching layer configured to have an acoustic impedance between a tissue to be imaged and a material of the transducer, a damping block configured to absorb ultrasound energy, and a processing circuit including a processor coupled to a memory device storing instructions thereon that, when executed, cause the processing circuit to perform operations including: determining, by a machine learning model, that the transducer is being navigated, dynamically adjusting, by the machine learning model, one or more acquisition parameters of the transducer to reduce an image quality of an image corresponding to the ultrasound signal, determining, by the machine learning model, that the transducer is positioned to capture a first ultrasound image, and dynamically readjusting, by the machine learning model, the one or more acquisition parameters to increase an image quality of the image corresponding to the ultrasound signal when the transducer is positioned to capture the first ultrasound image.

Another embodiment relates to a medical imaging system including: a processing circuit having a processor coupled to a memory device storing instructions thereon that, when executed, cause the processing circuit to perform operations including: determining, by a machine learning model, that a transducer of the medical imaging system is being navigated, wherein the transducer is configured to transmit and receive an ultrasound signal, dynamically adjusting, by the machine learning model, one or more acquisition parameters of the transducer to reduce an image quality of an image corresponding to the ultrasound signal, determining, by the machine learning model, that the transducer is positioned to capture a first ultrasound image, and dynamically readjusting, by the machine learning model, the one or more acquisition parameters to increase an image quality of the image corresponding to the ultrasound signal when the transducer is positioned to capture the first ultrasound image.

Another embodiment relates to method including: determining, by a machine learning model of a processing circuit of an ultrasound imaging system, that a transducer of the ultrasound imaging system is being navigated, wherein the transducer is configured to transmit and receive an ultrasound signal, dynamically adjusting, by the machine learning model of the processing circuit, one or more acquisition parameters of the transducer to reduce an image quality of an image corresponding to the ultrasound signal, determining, by the machine learning model of the processing circuit, that the transducer is positioned to capture a first ultrasound image, and dynamically readjusting, by the machine learning model of the processing circuit, the one or more acquisition parameters to increase an image quality of the image corresponding to the ultrasound signal when the transducer is positioned to capture the first ultrasound image.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a block diagram of an ultrasound imaging system, according to an example embodiment.

FIG. 2 is a block diagram of an artificial intelligence (AI) circuit used in the ultrasound imaging system of FIG. 1, according to an example embodiment.

FIG. 3 is a flow chart illustrating a method for rendering an ultrasound image for display, according to an example embodiment.

FIG. 4 is a flow chart illustrating a method for rendering an ultrasound image for display, according to another example embodiment.

FIG. 5 is a flow chart illustrating a method for rendering an ultrasound image for display using artificial intelligence, according to an example embodiment.

FIG. 6A is an illustration of an ultrasound image displayed while navigating to a diagnostic-quality image view, according to an example embodiment.

FIG. 6B is an illustration of a guidance system for positioning an ultrasound transducer to corresponding to the illustration of FIG. 6A, according to an example embodiment.

FIG. 7 is a flow chart illustrating a method for navigating an ultrasound transducer, according to an example embodiment.

FIG. 8 is a flow chart illustrating a method for optimizing an ultrasound image quality using artificial intelligence, according to an example embodiment.

FIG. 9 is a flow chart illustrating a method for re-optimizing an ultrasound image quality using artificial intelligence, according to an example embodiment.

### DETAILED DESCRIPTION

Referring generally to the figures, systems and methods for optimizing an image quality of an ultrasound image are shown and described. More specifically, the systems and methods described herein include utilizing a machine learning model to adjust imaging parameters of an ultrasound image based upon a determination of whether the ultrasound image is being captured.

Medical ultrasound imaging devices transmit acoustic energy and thermal energy into a patient's body by means of ultrasound waves and a temperature of a scan head (e.g., a probe), respectively. High-quality ultrasound images may require high amounts of acoustic and/or thermal energy to be transmitted. However, the amount of energy (e.g., mechanical/acoustic and/or thermal) that is permissible to be transmitted is regulated, thereby limiting the attainable imaging quality. For example, the temperature on a surface of a probe transducer that contacts a patient may not be permitted to be above a certain temperature (e.g., 42 degrees Celsius). A greater amount of energy that is transmitted to and from the probe may result in higher quality images due to an increased echo, but may also cause an increase in probe temperature. As such, probe temperature may also be a limiting factor.

For battery-operated wireless ultrasound probes, an available battery capacity poses another limiting factor. For example, when greater amounts of energy are transmitted to and from the probe, the probe consumes more battery energy, thereby permitting the probe to scan for shorter periods of time before a recharge or battery replacement is needed.

The systems and methods described herein provide a manner of maximizing acoustic power and transmitted energy at the time of a recorded ultrasound-image loop, while minimizing acoustic power and transmitted energy during the rest of the scan (e.g., while the probe is being maneuvered or navigated). For example, a significant portion of time spent performing an ultrasound scan is spent on navigating or setting up the probe to be positioned to achieve a good (e.g., diagnostic-quality) view. While the probe is being navigated to achieve this view (e.g., by adjusting the angle of the probe, etc.), a user (e.g., a sonographer) may not be interested in the ultrasound image being produced, as the view may not permit for adequate image analysis like a diagnostic-quality view can.

Additionally, in current ultrasound imaging systems, acquisition parameters are fixed for a selected imaging preset, leading to fixed image quality for the duration of a scan. Parameters may be changed manually by the user (e.g., sonographer) to optimize for patient variations (e.g., in height, weight, etc.). However, once optimized, imaging settings may be left unchanged for the duration of the actual scan session. This may be due to a human user requiring consistently high image quality to be able to navigate the ultrasound probe to a clinically usable view.

Additionally, ultrasound systems may utilize guidance tools powered by artificial intelligence (AI) that aid the user in navigating the ultrasound probe to achieve a clinically usable view. AI guidance tools may require or utilize a smaller amount of data relative to a human to analyze a current ultrasound image and generate guidance instructions that direct the user to the same clinically usable view. With such AI guidance tools, the user is relieved of the responsibility of navigating the probe (e.g., without aid). Further, when the AI systems are utilized, a maximum image quality may be used or required only when recording a sequence of ultrasound images for diagnostic purposes. Consequently, having fixed acquisition parameters may consume unnecessarily high amounts of an energy-transmission budget and wastes the limited battery capacity of wireless probes.

Therefore, the systems and methods described herein provide a manner of dynamically adjusting acquisition parameters of an ultrasound imaging system based on a quality metric provided by an AI guidance tool. For images that are far away from a clinically usable view (e.g., images obtained while the probe is being navigated), power consumption is reduced to a minimum of what is required for the AI tool to operate. This is achieved by adjusting imaging or acquisition parameters so that a lower-quality ultrasound image is rendered while the probe is being navigated and only the AI tool is analyzing the ultrasound images. Conversely, power (and hence image quality) is increased to a maximum when the image is approaching a clinically usable view. This is achieved by adjusting the imaging or acquisition parameters so that a higher-quality ultrasound image is rendered while the probe is positioned to capture a diagnostic-quality view that will be analyzed by a human. The systems and methods described herein may lead to increased image quality levels (e.g., beyond a limit of current systems) when diagnostically relevant (e.g., when an ultrasound image is being recorded), as well as increased overall scan times for battery operated probes.

Technically and beneficially, the systems and methods described herein provide longer scan times for wireless probes before requiring a recharge or battery change, due to lower average electric power consumption when ultrasound images that are not of a clinically usable view are rendered. Additionally, lower power consumption leads to less probe heating, and, therefore, a reduced need for the probe to throttle down to keep temperatures within regulatory limits. Currently, heating of the probe and a need to throttle down the probe to keep temperature within regulatory limits may limit an available scan time. This may reduce an attainable image quality as the probe temperature increases. Technically, and beneficially, with the systems and methods described herein, on average, a transmit power is reduced and can be intermittently increased (e.g., beyond a current maximum transmit power) when the AI tool determines that a clinically usable view is found. This may lead to an increase in image quality for recorded image sequences.

Before turning to the figures, which illustrate certain exemplary embodiments in detail, it should be understood that the present disclosure is not limited to the details or methodology set forth in the description or illustrated in the figures. It should also be understood that the terminology used herein is for the purpose of description only and should not be regarded as limiting.

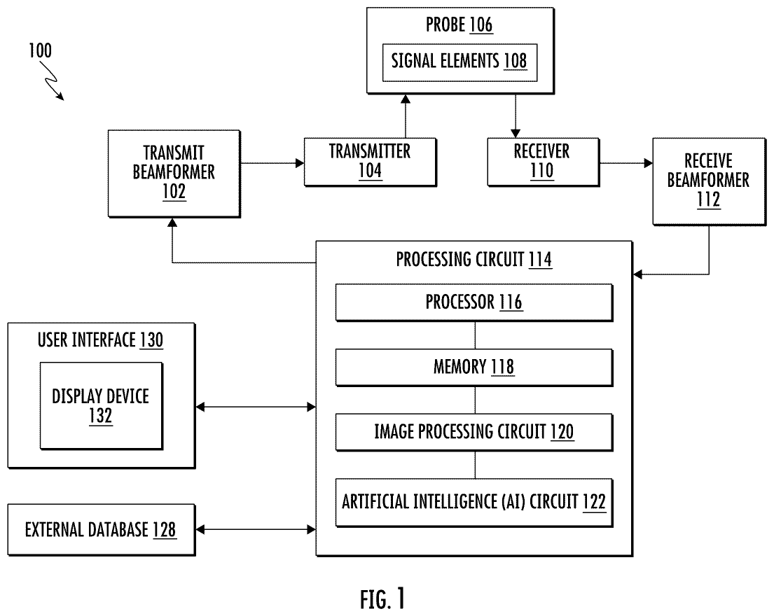

Referring to FIG. 1, a schematic diagram of an ultrasound imaging system 100 is shown. The ultrasound imaging system 100 may be used in a medical environment (e.g., hospitals, clinics, etc.), for example, by a sonographer, technician, or other clinician certified to collect ultrasound data from a patient.

An example of a procedure performed using the ultrasound imaging system 100 may be an echocardiogram. Echocardiograms are performed to detect heart abnormalities in a patient by collecting and processing ultrasound data (e.g., using the ultrasound imaging system 100, as described herein). During an echocardiogram, a sonographer follows a particular imaging protocol specific to echocardiography. The echocardiography-specific imaging protocol ensures that the heart is thoroughly captured by the ultrasound data and that the processing of the ultrasound data is focused on detecting heart abnormalities. The sonographer collects the ultrasound data by navigating a probe (e.g., probe 106, as described below) over the patient's chest until a sufficient volume of ultrasound images are collected. The collected images are stored in a central storage device (e.g., memory 118) and analyzed by the sonographer and/or an artificial intelligence system. Reference may be made throughout to a sonographer collecting images, adjusting imaging parameters, etc. However, it should be understood that an artificial intelligence model may be configured to perform any of the actions described as being performed by a sonographer. The sonographer generates a set of measurements from the images (e.g., 50-100 records), and the images and measurements are collectively reviewed by a cardiologist. The cardiologist provides any clinical findings/conclusions in a report submitted to the patient's medical record.

As shown in FIG. 1, the ultrasound imaging system 100 includes a transmit beamformer 102, a transmitter 104, a probe 106, a receiver 110, and a receive beamformer 112.

The transmit beamformer 102 may be either a hardware beamformer or a software beamformer. In embodiments where the transmit beamformer 102 is a hardware beamformer, the transmit beamformer 102 may include one or more of a graphics processing unit (GPU), a microprocessor, a central processing unit (CPU), a digital signal processor (DSP), or any other type of processor capable of performing logical operations. The transmit beamformer 102 may be configured to perform conventional beamforming techniques as well as techniques such as retrospective transmit beamforming (RTB). Alternatively, in embodiments where the transmit beamformer 102 is a software beamformer, a processor (e.g., processor 116, as described below) may be configured to perform some or all of the functions associated with the transmit beamformer 102.

The probe 106 may be a linear array probe, a curvilinear array probe, a sector probe, or any other type of probe configured to obtain two-dimensional (2D) B-mode data, 2D color flow data, M-mode data, three-dimensional (3D) data, four-dimensional (4D) data, or any other type of ultrasound data. Alternatively or additionally, the probe 106 may be any type of probe configured to obtain 2D B-mode data and data corresponding to another ultrasound mode that detects blood flow velocity in the direction of a vessel axis. In some embodiments, the probe 106 may include a position sensor configured to detect a position of the probe 106 relative to one or more reference locations. That is, the position sensor may continuously track movement (e.g., rotation, translation, orientation, etc.) of the probe 106 relative to the location of the probe 106 when the anatomy being imaged is identified. For example, the anatomy being imaged may be identified as a left atrial appendage (LAA) at a first location of the probe 106. Then, the position sensor may track the movement of the probe 106 relative to the LAA in order to identify successive locations of the probe 106. In some embodiments, the position sensor may transmit position data to be stored within the ultrasound imaging system 100 (e.g., in memory 118).

The probe 106 may include a transducer configured to transmit and receive an ultrasound signal. In some embodiments, as shown in FIG. 1, the probe 106 includes signal elements 108. The signal elements 108 may be arranged in a transducer array, and in some embodiments may be arranged in a one-dimensional (1D) or 2D array. The transmit beamformer 102 and the transmitter 104 drive the signal elements 108 to emit pulsed ultrasonic signals into a body of a subject (e.g., a patient). For example, during an echocardiogram, a sonographer or other clinician may navigate the probe 106 over a patient's chest so that the signal elements 108 in the probe 106 emit the pulsed ultrasonic signals into the patient's thoracic cavity. The pulsed ultrasonic signals are then back-scattered from anatomical structures in the body, such as blood cells or muscular tissues, to produce echoes that return to the signal elements 108. That is, the signal elements 108 may include the transducer configured to transmit and receive the ultrasound signal, a matching layer configured to prevent an acoustic impedance mismatch between a tissue to be imaged and a material of the transducer (e.g., such that the pulsed electronic signals can be back-scattered from the anatomical structures in the body and received as echoes by the signal elements 108 and prevent unwanted scattering and/or reflection of the pulsed electronic signals at a tissue-transducer interface), and a damping block configured to absorb ultrasound energy.

The receiver 110 receives the echoes from the probe 106 and converts the echoes into electrical signals. The electrical signals are then passed through the receive beamformer 112, which produces the ultrasound data from the electrical signals. As described above with reference to the transmit beamformer 102, the receive beamformer 112 may be either a hardware beamformer or a software beamformer. In embodiments where the receive beamformer 112 is a hardware beamformer, the receive beamformer 112 may include one or more of a GPU, a microprocessor, a CPU, a DSP, or any other type of processor capable of performing logical operations. The receive beamformer 112 may be configured to perform conventional beamforming techniques as well as techniques such as retrospective transmit beamforming (RTB). Alternatively, in embodiments where the receive beamformer 112 is a software beamformer, a processor (e.g., processor 116, as described below) may be configured to perform some or all of the functions associated with the receive beamformer 112.

Although the transmit beamformer 102, the transmitter 104, the receiver 110, and the receive beamformer 112 are shown in FIG. 1 as being components of the ultrasound imaging system 100 that are distinct from the probe 106, it should be appreciated that in some embodiments, the probe 106 may include electronic circuitry configured to perform the functions of each of the transmit beamformer 102, the transmitter 104, the receiver 110, and/or the receive beamformer 112. That is, all or part of the transmit beamformer 102, the transmitter 104, the receiver 110, and/or the receive beamformer 112 may be situated within the probe 106.

Referring still to FIG. 1, the ultrasound imaging system 100 is shown to include a processing circuit 114. As shown, the processing circuit 114 may include at least one processor 116, a memory 118, an image processing circuit 120, and an artificial intelligence (AI) circuit 122. In this way, the processing circuit 114 may be structured or configured to execute or implement the instructions, commands, and/or control processes described herein with respect to the processor 116, the memory 118, the image processing circuit 120, and the AI circuit 122. While shown as being separate from the probe 106 in FIG. 1, it will be appreciated that the processing circuit 114 can be part of the probe 106. For example, the processing circuit 114 can be disposed in a handheld housing of the probe 106 (e.g., in the case of the probe 106 being a wireless probe).

The processor 116 may include a CPU, a GPU, a microprocessor, a DSP, a general-purpose single-or multi-chip processor, a field-programmable gate array (FPGA), or any other type of processor capable of performing logical operations. A general-purpose processor may be a microprocessor, or, any conventional processor, or state machine. A processor also may be implemented as a combination of computing devices, such as a combination of a DSP and a microprocessor, a plurality of microprocessors, one or more microprocessors in conjunction with a DSP core, or any other such configuration. In some embodiments, the processor 116 may be shared by multiple circuits (e.g., the circuits of the processor 116 may include or otherwise share the same processor which, in some example embodiments, may execute instructions stored, or otherwise accessed, via different areas of the memory 118). Alternatively or additionally, the processor 116 may be structured to perform or otherwise execute certain operations independent of one or more co-processors. In some embodiments, two or more processors may be coupled via a bus to enable independent, parallel, pipelined, or multi-threaded instruction execution. All such variations are intended to fall within the scope of the present disclosure.

The processor 116 may be configured to control the transmit beamformer 102, the transmitter 104, the receiver 110, and the receive beamformer 112. The processor 116 may also be in electronic communication with the probe 106. For purposes of this disclosure, the term “electronic communication” may be defined to include both wired and wireless communications.

In some embodiments, the processor 116 may be configured to control the probe 106 during data acquisition. That is, the processor 116 may control the data acquisition by controlling which of the signal elements 108 are active and by controlling a shape of the beam emitted from the probe 106. Alternatively or additionally, the processor 116 may include a complex demodulator configured to demodulate radio frequency (RF) data obtained by the probe 106 and generate raw data. According to other embodiments, the demodulation of the RF data may be performed by another component of the ultrasound imaging system 100. The processor 116 may perform the processing operations described herein according to a plurality of selectable ultrasound modalities.

Depending on the mode of operation of the ultrasound imaging system 100, the processor 116 may process ultrasound data obtained by the probe 106 according to the mode of operation to generate 2D or 3D image data. For example, the mode of operation may include B-mode, color flow Doppler mode, M-mode, color M-mode, spectral Doppler, elastography, TVI, strain, strain rate, and the like. Various of these modes of operation may be configured to, for instance, convert ultrasound data from beam space coordinates (e.g., received from the receive beamformer 112) to display space coordinates (e.g., such that the ultrasound data may be displayed as image data). In some embodiments, the mode of operation may allow for video processing by the processor 116 such that a series of images (e.g., processed ultrasound data) may be displayed in real-time while a scanning session/procedure is being performed on a patient. An operator of the ultrasound imaging system 100 (e.g., a sonographer) may switch between various modes in order to obtain a variety of ultrasound data and to perform a complete scan of an anatomical region of interest. For example, the operator may switch between modes using user interface 130 (e.g., using physical controls, interface inputs representing physical controls, etc.). While the term “image” or “images” are used herein to for the purposes of example, it will be appreciated that such terms cover still images as well as videos, clips, or a series of images for each. For example, in some embodiments, the image or images may include a 1-10 second clip derived from the image data.

The processor 116 performs the processing operations in real-time as the echo signals are received by the receiver 110 from the probe 106. For the purposes of this disclosure, the term “real-time” is defined to include a procedure that is performed without any intentional delay. As an illustrative, non-limiting example, in certain instances, the ultrasound imaging system 100 may obtain images at a real-time volume-rate of 7-20 volumes/sec. It should be appreciated, however, that the real-time volume-rate may be dependent on the length of time that it takes to obtain each volume of data for display. Thus, the ultrasound imaging system 100 may be configured to obtain 2D data of an anatomical region at a faster rate than 3D data of the same anatomical region because it takes longer to obtain a volume of 3D data than the same volume of 2D data. Similarly, when the ultrasound imaging system 100 obtains a relatively large volume of data, the real-time volume-rate may be slower than for a smaller volume of data. For example, during an abdominal scan, the real-time volume-rate may be slower if the patient is an adult versus if the patient is an infant because the volume of data is larger for the adult than for the infant (e.g., due to the abdomen of an adult being larger than the abdomen of an infant). Therefore, certain implementations of the ultrasound imaging system 100 may have real-time volume-rates that are faster than 20 volumes/sec, while other implementations of the ultrasound imaging system 100 may have real-time volume-rates that are slower than 7 volumes/sec.

In some embodiments, the ultrasound imaging system 100 may include multiple processors configured to perform the processing operations/functionality described with reference to processor 116. For example, in such embodiments, a first processor of the multiple processors may be configured to demodulate and decimate the RF signal while a second processor of the multiple processors may be configured to further process the RF data prior to displaying an image representative of the data. It should be appreciated that other embodiments may use a different arrangement of processors.

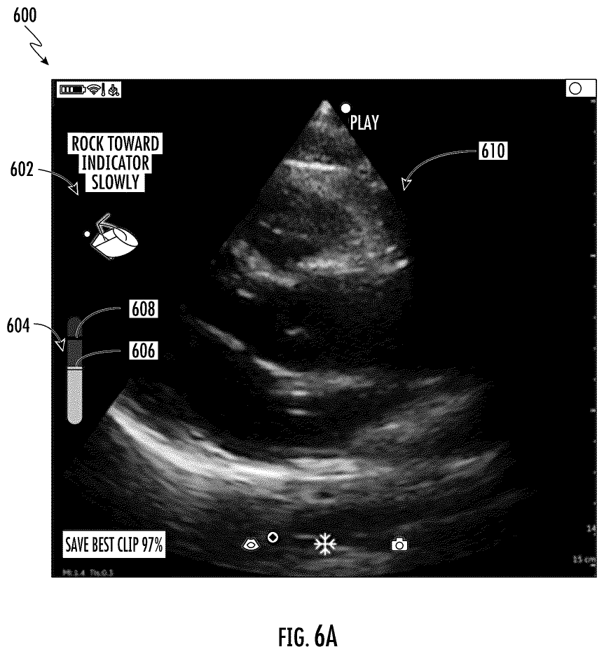

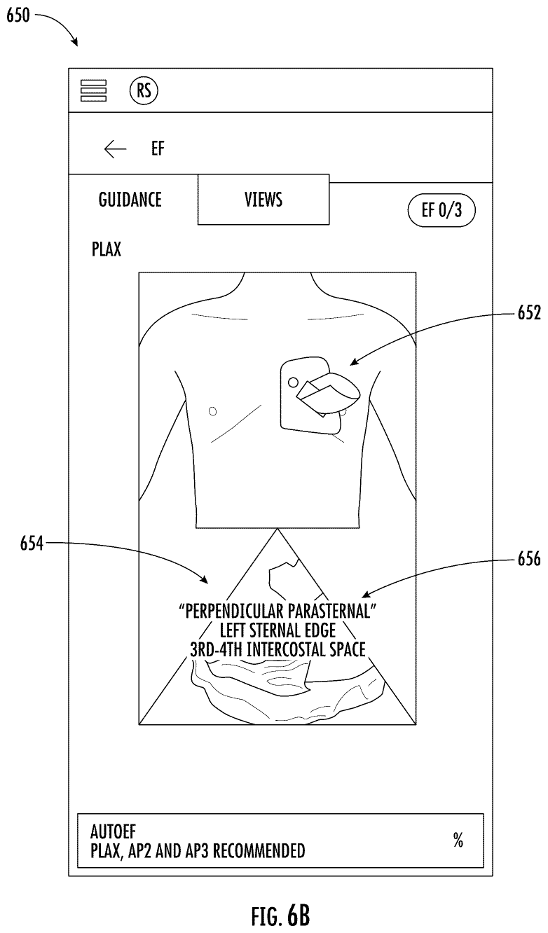

The processor 116 may also be in electronic communication with the display device 132 such that the processor 116 may process ultrasound data obtained by the probe 106 and generate images to display on the display device 132 (e.g., ultrasound image 600, as described below with reference to FIG. 6A).

As shown in FIG. 1, the processing circuit 114 also includes the memory 118. The memory 118 may be configured to, for example, store processed volumes of data obtained by the ultrasound imaging system 100 (e.g., ultrasound data collected by the probe 106, user inputs received by the user interface 130, etc.). For example, the memory 118 may be a hospital picture archiving and communication system (PACS). The memory 118 (e.g., memory, memory unit, storage device, etc.) may include one or more devices (e.g., RAM, ROM, Flash memory, hard disk storage, etc.) for storing data and/or computer code for completing or facilitating the processes, layers, and modules described in the present application. The memory 118 may be or include tangible, non-transient volatile memory or non-volatile memory. The memory 118 may also include database components, object code components, script components, or any other type of information structure for supporting the activities and information structures described in the present application.

In various embodiments, the memory 118 may have varying capacity (e.g., storage space) across embodiments of the ultrasound imaging system 100. For example, the memory 118 may be configured to store at least 60 minutes'worth of ultrasound data. The ultrasound data may be stored in the memory 118 such that the ultrasound data may be retrieved according to an order/time of acquiring the data. That is, the ultrasound data may be stored with a timestamp indicating a time at which the ultrasound data was collected and may be retrieved starting with an oldest time at which the ultrasound data was collected.

The processing circuit 114 also includes the image processing circuit 120 and the AI circuit 122. Both the image processing circuit 120 and the AI circuit 122 are configured to facilitate providing recommended imaging parameters during an ultrasound scan, as described herein.

The image processing circuit 120 is configured to receive image data obtained by the transducer of the probe 106 during an ultrasound scan. The image data refers to ultrasound data collected by the probe 106 while performing an ultrasound examination on a patient. For example, the image data may be collected during a fetal ultrasound and may therefore include various images of a patient's uterus and the fetal anatomy contained therein. As another example, the image data collected during an echocardiogram may include images of a patient's heart and specific structures (e.g., ventricles, atria, etc.) therein. The image processing circuit 120 may include multiple deep learning-based models configured to analyze the image data. For example, the image processing circuit may be configured to identify a view from which the image data is captured, an anatomical structure or other feature captured by the image data, the presence of a pathology in the image data, and so on. The image processing circuit 120 may be configured to identify the anatomical structure using one or more algorithms (e.g., image processing algorithms such as edge detection, machine learning models, deep neural networks, etc.). In some embodiments, the image processing circuit 120 may identify anatomical features such as bones, blood vessels, organs, etc., based on a shape, relative proximity, apparent depth, orientation, etc. of said features in the image data.

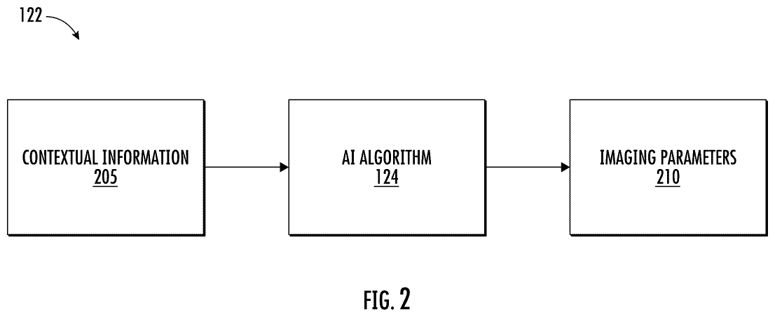

As described in greater detail below, the AI circuit 122 may be configured to adjust or modulate imaging parameters (e.g., imaging parameters 210, as shown in FIG. 2) based on contextual information (e.g., contextual information 205, as shown in FIG. 2) regarding a medical imaging procedure. For example, during an ultrasound performed using the ultrasound imaging system 100, the AI circuit 122 may be configured to adjust at least one of a transmission rate (e.g., an acquisition rate or a frame rate) or an ultrasound image resolution frequency based on a determination of whether the probe 106 is being navigated to a position in which a diagnostic-quality ultrasound image can be captured or is already positioned in which a diagnostic-quality ultrasound image can be captured. As described herein “capturing” an ultrasound image may refer to the act of storing the ultrasound image (e.g., in the memory 118).

The ultrasound imaging system 100 may also include an external database 128 and a user interface 130. The external database 128 refers to a database from which the processing circuit 114 (e.g., the AI circuit 122) may retrieve information used to provide recommended imaging parameters during an ultrasound scan. For example, the external database 128 may be a medical information database. The medical information database may store clinical guidelines, standard practices, medical literature, medical textbooks, published research, previous case studies, and so on. Depending on an implementation of the ultrasound imaging system 100 and/or a procedure performed thereby, the AI circuit 122 may retrieve clinical guidelines, standard practices, medical literature, medical textbooks, published research, and previous case studies related to the implementation and/or procedure. For example, if the ultrasound imaging system 100 is being used in a hospital setting to perform an LAA closure procedure, the AI circuit 122 may retrieve clinical guidelines and standard practices related to the hospital setting and the LAA closure procedure. Continuing with this example, the AI circuit 122 may also retrieve information from the medical literature, medical textbooks, published research, and previous case studies related to cardiac anatomy and the LAA closure procedure.

The user interface 130 may be used by a sonographer or other clinician to control operation of the ultrasound imaging system 100. For example, the sonographer may use the user interface 130 to control the input of patient data, to change a scanning or display parameter, to adjust a segmentation of an anatomical feature depicted in an ultrasound image, and/or to select various other modes, operations, parameters, etc. of the ultrasound imaging system 100. In some embodiments, the user interface 130 may include an off-the-shelf consumer electronic device such as a smartphone, a tablet, a laptop, and so on. For the purposes of this disclosure, the term “off-the-shelf consumer electronic device” is defined to be an electronic device that was designed and developed for general consumer use and one that was not specifically designed for use in a medical environment. Alternatively, in other embodiments, the user interface 130 may be an electronic device that was designed and developed for use in a medical environment.

According to some embodiments, the user interface 130 may be physically separate from the rest of the ultrasound imaging system 100 (e.g., the transmit beamformer 102, the transmitter 104, the probe 106, the receiver 110, the receive beamformer 112, the processing circuit 114, and/or the external database 128). The user interface 130 may communicate with the processor 116 through a wireless protocol, such as Wi-Fi, Bluetooth, wireless local area network (WLAN), near-field communication, and so on. According to some embodiments, the user interface 130 may communicate with the processor 116 through an application programming interface (API).

In some embodiments, the user interface 130 may include physical controls such as one or more of buttons, sliders, a rotary knob, a mouse, a keyboard, a trackball, hard keys linked to specific actions, soft keys that may be configured to control different functions, and so on. As shown in FIG. 1, the user interface 130 may also include a display device 132. In some embodiments, the display device 132 may be configured to display a graphical user interface (GUI) based on an instruction from the memory 118. The GUI may include user interface icons representing commands and instructions relating to the operation of the ultrasound imaging system 100. The user interface icons of the GUI may be configured such that a user (e.g., the sonographer, clinician, etc.) may select a specific user interface icon in order to initiate a specific function controlled by the GUI. For example, various user interface icons may be used to represent windows, menus, buttons, cursors, scroll bars, and so on. That is, the physical controls of the user interface 130 may be included as individual hardware elements, as user interface icons displayed on the display device 132, or as a combination of hardware elements and user interface icons.

In some embodiments, the display device 132 may include a touch-sensitive display device or a touch screen. According to such embodiments, the touch screen may be configured to interact with the GUI displayed by the display device 132 such that a user (e.g., the sonographer) can interact with the GUI via the touch screen. The touch screen may be a single-point touch screen that is configured to detect a single contact point at a time, or the touch screen may be a multi-point touch screen that is configured to detect multiple points of contact at a time. For embodiments where the touch screen is a multi-point touch screen, the touch screen may be configured to detect multi-point gestures involving contact from two or more of a user's fingers at a time. The touch screen may be a resistive touch screen, a capacitive touch screen, or any other type of touch screen that is configured to receive inputs from a stylus or one or more of a user's fingers. According to some embodiments, the touch screen may be an optical touch screen that uses technology such as infrared light or other frequencies of light to detect one or more points of contact initiated by a user. In some embodiments, the touch screen may be incorporated as part of the display device 132 or may be separate from the display device 132. The user interface 130 may also include a proximity sensor configured to detect objects and/or gestures that are within a predetermined distance (e.g., five feet, six inches, ten centimeters, etc.) of the proximity sensor. In various embodiments, the proximity sensor may be located on the display device 132 or as part of a touch screen that is separate from the display device 132.

Referring now to FIG. 2, the AI circuit 122 of the ultrasound imaging system 100 is shown in greater detail. The AI circuit 122 is configured to determine a quality of an ultrasound image corresponding to a current position of the probe 106. The quality may be expressed as a quality score (e.g., a score on a range of 0-100, where 100 corresponds to an optimal, diagnostic-quality image) that indicates a relative quality of the image generated using the current imaging parameters 210 and probe 106 position. The term “quality” when used in reference to a quality score may refer to how close the probe is positioned to obtaining a diagnostic-quality view of a desired anatomical structure. It should be understood that the quality score can be expressed as any value and/or on any scale, including a percentage value, a continuous spectrum, a threshold-based score, etc. In some embodiments, the quality score is determined by comparing the image to an image library including a plurality of historical ultrasound images. The image can be compared to historical ultrasound images exceeding a minimum image quality score (e.g., for a particular image to be captured, as part of guidance instructions for a particular diagnosis, etc.) to determine if the transducer is in position to obtain an ultrasound image exceeding the minimum quality requirements, which then triggers the transducer to increase an output of acoustic and/or thermal energy to obtain higher quality image data. In some embodiments, the quality score is determined by a machine learning model.

The AI circuit 122 is further configured to adjust imaging parameters 210 based on whether the probe 106 is currently in a position to capture a diagnostic-quality image or is being navigated (e.g., to arrive at a position to capture a diagnostic-quality image). The AI circuit 122 may generate guidance instructions that direct a user (e.g., a sonographer) to obtain a clinically usable view or ultrasound image. In some embodiments, the guidance instructions are based on clinic guidelines for obtaining ultrasound images for determining a particular diagnosis. Thus, because the sonographer is not relying on their own view of the ultrasound image as they navigate the probe 106 to arrive at the clinically usable view, a reduced-quality image can be generated or displayed as the user navigates the probe according to the instructions generated by the AI circuit 122. For example, while the probe 106 is being navigated into a position to capture an ultrasound image according to instructions generated by the AI circuit 122, the sonographer may not be relying on the displayed ultrasound image to determine whether an acceptable view is being achieved. As such, in order to reduce battery consumption of the probe 106, thereby extending a battery life of the probe 106, the ultrasound image displayed on the display device 132 while the probe 106 is being navigated may be reduced in quality via the AI circuit 122. Thus, when the quality score is below a threshold value, the generated ultrasound images may be displayed live on the display device 132 and/or analyzed by the AI system 122.

As shown, the AI circuit 122 receives contextual information 205 regarding an ultrasound scan. In some embodiments, the contextual information 205 may include an indication of whether the probe 106 is currently positioned such that a diagnostic-quality image of the anatomy can be captured or whether the probe 106 is currently being navigated (e.g., by the sonographer) to obtain a diagnostic-quality image in the future. For example, the contextual information 205 may therefore include a direction of the probe 106, a current location or position of the probe 106, and/or current settings of imaging parameters such as a frequency, an acquisition angle, a dynamic power, a gain, a compound imaging setting, a transmission rate, an acquisition rate, a frame rate, or an ultrasound image resolution. The contextual information 205 may also include a quality score of an ultrasound image.

The contextual information 205 may be used as an input to an AI algorithm 124. In some embodiments, the AI algorithm 124 may be a Bayesian neural network. As shown in FIG. 2, the AI algorithm 124 is configured to generate updated imaging parameters 210 based on the received contextual information 205. The imaging parameters 210 may include, for example, at least one of a frequency, an acquisition angle, a dynamic power, a gain, a compound imaging setting, a transmission rate, an acquisition rate, a frame rate, or an ultrasound image resolution, and so on, to use for image acquisition during an ultrasound scan. The imaging parameters 210 may be the same type or types of imaging parameters as those used as contextual information 205. In an example implementation of the AI circuit 122 shown in FIG. 2, if the contextual information 205 includes an indication that the probe 106 is being navigated (e.g., the probe is not positioned to obtain a diagnostic-quality image view) and the current imaging parameters are causing a high quality ultrasound image to be displayed, the AI algorithm 124 may determine that the quality of the ultrasound image can be reduced (e.g., by adjusting imaging parameters) until the probe 106 has being navigated to the location where a clinically usable view is achieved. As used herein, a “high quality ultrasound image” or “high quality image” may refer to an image generated using imaging parameters above a certain threshold value (e.g. above a certain frame rate, above a certain acquisition rate, above a certain resolution, etc.). A high quality image may be obtained when a quality score generated by the AI circuit 122 is above a threshold value. Similarity, as used herein, a “low quality ultrasound image” or “low quality image” may refer to an image generated using imaging parameters below a certain threshold value (e.g. below a certain frame rate, below a certain acquisition rate, below a certain resolution, etc.). A low quality image may be obtained when a quality score generated by the AI circuit 122 is below a threshold value. Upon determining that the quality of the ultrasound image can be reduced, as may be indicated by the quality score of the image, the AI algorithm 124 may adjust one or more imaging parameters 210 to reduce a quality of the ultrasound image (e.g., such that the imaging parameters 210 are below a threshold value).

In some embodiments, the AI algorithm 124 may determine that the quality of the ultrasound image can be increased (e.g., by adjusting imaging parameters) because the probe 106 has arrived at a position where an image is to be captured (e.g., the probe 106 is no longer being navigated), as may be indicated by a quality score of the image. Upon determining that the quality of the ultrasound image can be increased, the AI algorithm 124 may adjust one or more imaging parameters 210 to increase a quality of the ultrasound image (e.g., such that the imaging parameters 210 are above a threshold value). For example, based on a determination that the quality score is at or above a threshold value, the AI system 122 may automatically trigger capturing of the generated ultrasound image and storing the ultrasound image (e.g., stored in the memory 118, recorded to a disk, etc.).

In some embodiments, the AI algorithm 124 may be trained using information regarding historic ultrasound scans performed by expert sonographers (e.g., sonographers with specific qualifications, sonographers having a number of years of experience, etc.). For example, an expert sonographer may conduct an echocardiogram or other ultrasound (e.g., a fetal ultrasound, etc.). The parameters regarding the echocardiogram (e.g., the imaging parameters used while the probe 106 is being navigated to the diagnostic-quality view, the imaging parameters used when the probe 106 is at the diagnostic-quality view, an AI score for the images during navigation and during image capture, etc.) may be stored (e.g., in the memory 118) as contextual information 205. Furthermore, the imaging parameters used by the expert sonographer during the echocardiogram or other ultrasound may be stored (e.g., in the memory 118) as imaging parameters 210 corresponding to the contextual information 205. In some instances, the imaging parameters used by the expert sonographer when the probe 106 is positioned to capture a diagnostic-quality image (e.g., when the probe is not being navigated) refer to the acquisition parameters of an ultrasound probe (e.g., probe 106) at a moment when the image quality is accepted by the expert sonographer or the AI circuit 122 (e.g., when the expert sonographer instructs the ultrasound imaging system 100 to “freeze” and/or when the AI circuit 122 has navigated the probe 106). In other instances, the imaging parameters used by the expert sonographer when the probe 106 is being navigated refer to the acquisition parameters of an ultrasound probe (e.g., probe 106) at one or more moments when the image quality is not accepted by the expert sonographer or the AI circuit 122 (e.g., when the expert sonographer is moving the probe according to guidance instructions from the AI circuit 122). The AI algorithm 124 may be trained using this information regarding the historic ultrasound scan such that when the contextual information 205 regarding an ultrasound scan includes at least one of the probe 106 being navigated, the probe 106 being at a position corresponding to a diagnostic-quality image, the imaging parameters being above or below a threshold value, and/or a quality score being above or below a threshold value, the AI algorithm 124 may be configured to adjust or modulate imaging parameters 210 based on the imaging parameters 210 used by the expert sonographer when conducting an ultrasound scan having the same contextual information 205.

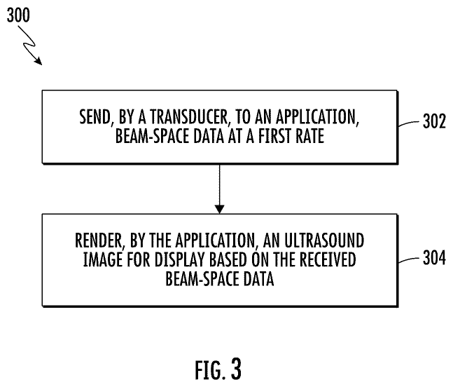

Referring now to FIG. 3, a method 300 for rendering an ultrasound image for display is shown, according to an example embodiment. The method 300 may illustrate a method of rendering an ultrasound image without the use of artificial intelligence (e.g., without the use of the AI circuit 122).

At process 302, the probe 106 (e.g., a transducer of the probe 106) may send or transmit beam-space data to an application (e.g., an application of the display device 132). The beam-space data may be associated with a fixed number of beams and/or samples. For example, to render or display any ultrasound image, the probe 106 may transmit beam-space data using the same number of beams and/or samples (e.g., 200 beams×600 samples). The probe 106 may send a maximum or largest amount of beam-space data possible to the application at all times. For example, the probe 106 may determine the largest amount of beam-space data that can be transmitted to the application while balancing image quality, power consumption, and a temperature of the probe 106 (e.g., so the probe 106 does not exceed a threshold temperature value). In traditional scanning methods (e.g., those described by the method 300), the ultrasound images may be viewed or consumed only by a human observer (e.g., a sonographer). As such, continuous maximum image quality may be needed, thereby causing the probe 106 to send the largest amount of beam-space data possible.

At process 304, the application (e.g., of the display device 132) receives the beam-space data and renders an ultrasound image for display. The nature of the received beam-space data (e.g., that the largest amount of beam-space data is transmitted) may cause the application to render a high quality image (e.g., an image above a certain resolution). For example, the application of the display device 132 may render the image at a resolution of 1000 pixels×1000 pixels. Transmitting large amounts of data and rendering a high quality ultrasound image at all times may cause the probe 106 to utilize a large amount of battery (e.g., when the probe 106 is configured as a handheld probe).

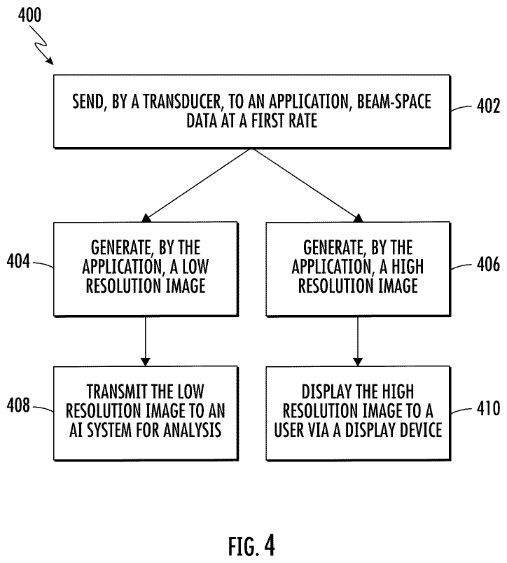

Referring now to FIG. 4, a method 400 for rendering an ultrasound image for display is shown, according to another example embodiment. As an example, the method 400 may refer to a method that utilizes AI-powered guidance to navigate to a diagnostic-quality image. For example, the method 400 may describe a guidance operation for a cardiac ultrasound. However, it should be understood that the method 400 may be used during a guidance operation for any type of ultrasound (e.g. fetal, etc.).

At process 402, the probe 106 (e.g., a transducer of the probe 106) sends or transmits beam-space data to an application of the display device 132. The beam-space data may be sent to the application at a first rate. The first rate may be a rate that causes the maximum amount of data to be transmitted (e.g., 200 beams×600 samples) while balancing image quality, power consumption, and a temperature of the probe 106.

At process 404, the application of the display device 132 generates a low resolution image using the transmitted (and subsequently received) beam-space data. For example, the application may generate a low quality image that has a resolution of 256 pixels×256 pixels.

At process 406, the application of the display device 132 generates a high resolution image using the transmitted (and subsequently received) beam-space data. For example, the application may generate a high quality image that has a resolution of 1000 pixels×1000 pixels.

At process 408, the application of the display device 132 transmits the low resolution image generated at process 404 to an artificial intelligence system (e.g., the AI circuit 122). The AI circuit 122 may analyze the low resolution image. For example, the AI circuit 122 may analyze the image to determine whether the probe 106 is positioned at a location to capture a diagnostic-quality image or whether the probe 106 is to continue to be navigated. The AI circuit 122 may further analyze the low resolution image to determine a subsequent guidance instruction or step to display to the user (e.g., sonographer) to continue navigating the probe 106. The AI circuit 122 may be able to successfully perform an image analysis with a low resolution input image. For example, a human eye may be unable to adequately analyze an ultrasound image having a resolution of 256 pixels×256 pixels, but the AI circuit 122 may be able to adequately analyze such an image.

At process 410, the application of the display device 132 displays the high resolution image generated at process 406 to a user (e.g., a sonographer) via the display device 132. As stated above, a human eye may be unable to adequately analyze an ultrasound image having a low resolution (e.g., 256 pixels×256 pixels). For example, a human may be unable to properly identify different structures or anatomy within an ultrasound image if the resolution is not sufficiently high. Additionally, changing a resolution of the images rendered by the application may not affect power consumption and heating of the probe 106 (e.g., because the probe 106 continues to transmit a maximum amount of beam-space data). Further, sampling that occurs on the probe 106 may not affect power consumption of the probe 106, but a pulse generated by the probe 106 may affect power consumption.

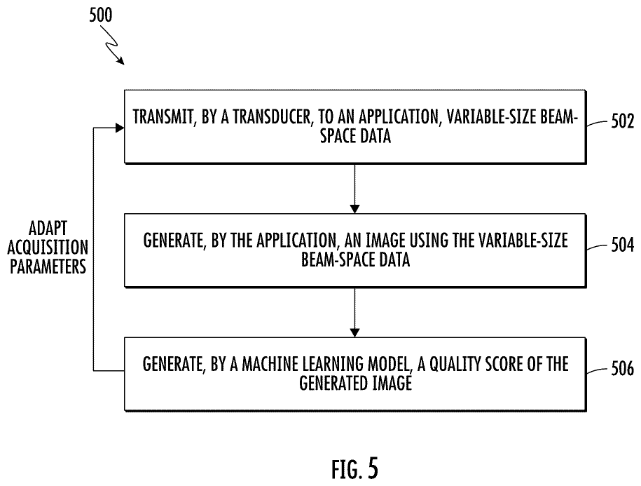

Referring now to FIG. 5, a method 500 for rendering an ultrasound image for display using artificial intelligence is shown, according to an example embodiment. As will be described, an artificial intelligence model (e.g., the AI circuit 122) may generate an output used to dynamically adapt acquisition parameters rather than using fixed acquisition parameters in the probe 106.

At process 502, the probe 106 (e.g., a transducer of the probe 106) transmits variable-size beam-space data to an application (e.g., an application of the display device 132). As will be described herein, the size of the beam-space data may depend upon a quality score of a previously-generated ultrasound image. Variable-size beam-space data may mean that, for each ultrasound image generated by an application, the data used to generate the image may be of a different size (e.g., a different number of beams and/or samples), thereby affecting the quality (e.g., resolution, frame rate, etc.) of the image. For example, when an image is rendered for display to a user, the beam-space data may be oversampled, thereby generating an image that includes more pixels than there are beam-space samples, causing an increased image quality.

At process 504, the application generates an image using the variable-size beam-space data. Depending on the size of the beam-space data transmitted by the probe 106, the resolution or other parameter, such as frame rate, of the generated image may be high (e.g., above a threshold value) or low (e.g., below a threshold value).

At process 506, a machine learning model (e.g., the AI algorithm 124) generates a quality score of the image generated at process 504. The quality score may be an assessment of the diagnostic quality of the generated image resulting from the probe position. That is, the quality score may be based on which anatomy or portion(s) of anatomy are visible in the generated image (e.g., an image has a higher quality score when the probe is positioned such that the desired anatomy to be imaged is visible in the generated image). For example, the quality score may be a whole number between 0 and 100 that indicates a quality of the image relative to an ability of the image to be clinically usable. For example, a quality score of 0 may indicate that the image has such poor quality (e.g., a low resolution, etc.) that the image is unable to be used for diagnostic purposes. A quality score of 100 may indicate that the image has a maximum resolution and the image is an optimal image for clinical use (e.g., for diagnoses, etc.).

When navigating the probe 106, a lower image quality may be sufficient, as the AI circuit 122 may be able to adequately analyze the image at a lower resolution (or other parameter(s)) compared to a human. When the probe 106 is positioned to capture an image, a higher image quality may be utilized, since a human may then be analyzing the image and capturing the image.

Based on the generated quality score, the AI circuit 122 adapts acquisition or imaging parameters and the method 500 repeats. For example, when the AI circuit 122 determines that the quality score is below a threshold value, the AI circuit 122 may determine that the probe 106 is not near or approaching a clinically-usable view, and can adjust acquisition or imaging parameters to reduce the quality of a subsequently-generated image. Reducing acquisition or imaging parameters (e.g., reducing a frame rate) may alter the size of the beam-space data that is transmitted at process 502.

As the probe moves toward a clinically-usable view, the quality score generated at process 506 may increase. As such, upon a determination at process 506 that the quality score has increased (e.g., relative to a previous quality score) or is above a threshold value, the AI circuit 122 may adjust the acquisition parameters by increasing the parameters (e.g., increasing a frame rate relative to a frame rate used to generate a previous image). This may increase the size of the beam space data transmitted at process 502.

In some embodiments, the AI circuit 122 may increase the acquisition parameters responsive only to a determination that an image is to be captured. For example, the probe 106 may be positioned to capture a clinically-usable view for a certain period of time (e.g., 3 minutes), meaning that the quality score may be above the threshold value for that period of time. However, the sonographer may only capture a five second video or clip of the clinically-usable view. As such, rather than increasing the image quality for the entirety of the 3 minute duration (e.g., the entirety of the time the quality score is above the threshold value), thereby consuming battery power of the probe, which may be unnecessary, the AI circuit 122 may only increase the imaging parameters responsive to a determination that the image is being captured. For example, the sonographer may interface with the display device 132 in such a way that indicates that the ultrasound image is to be captured. Responsive to a determination that the image has been captured, the AI circuit 122 may reduce the image quality, even though the quality score may still indicate that the probe 106 is positioned to capture a clinically usable view.

This manner of operation may also prevent a temperature of the probe from exceeding the regulatory limit, thereby permitting higher quality images to be captured, because the imaging parameters are increased for a shorter period of time. For example, acquisition parameters of the probe 106 may be automatically throttled down (e.g., reduced) responsive to a determination that the probe temperature has exceeded a regulatory limit, thereby causing lower-quality images to be rendered while reducing a temperature of the probe. The AI circuit 122 may determine (e.g., via a quality score) that the probe is approaching a diagnostic-quality view, and may adjust acquisition parameters to render the highest-quality image possible. The AI circuit 122 may adjust the parameters responsive to a determination that the sonographer is to capture the ultrasound image. An average deposition of thermal energy onto the patient may be lower using adaptive tuning of imaging parameters via the AI circuit 122. As such, the probe temperature may operate below a regulatory limit when the AI circuit 122 does not actively tune imaging parameters. This lower average probe temperature may allow short spikes in the amount of thermal energy deposited on a patient without exceeding the regulatory limit. In this manner, downward throttling of the parameters may be delayed until an image of sufficient quality of obtained. Once the image has been captured, downward throttling may resume to reduce the probe temperature.

Referring now to FIG. 6A, an illustration of an ultrasound image 600 displayed while navigating to a diagnostic-quality image view is shown, according to an example embodiment.

As shown, the ultrasound image 600 includes guidance instructions 602, a quality score indicator 604, and an image 610. As a user (e.g., sonographer) moves the probe 106, the image 610 may change. The image 610 may change in both view (e.g., what is shown) and quality.

The ultrasound image 600 may specifically show a guidance tool that uses AI systems to guide a user to a diagnostic-quality ultrasound view. As such, the ultrasound image 600 may include guidance instructions 602. The guidance instructions 602 may include written instructions that direct or guide a user on how to move the probe 106 and/or a graphical or pictographic representation of the guidance. For example, as shown in FIG. 6A, the written guidance states “rock toward indicator slowly.” The graphical guidance includes an image that corresponds to the written guidance and shows a pictographic representation of rocking the probe toward the indicator slowly.

As the user moves the probe according to the guidance instructions 602, the image 610 may change and, as a result, the quality score indicator 604 may change as well. As shown, the quality score indicator 604 is a bar. A portion 606 of the quality score indicator 604 may move as the image 610 changes, corresponding to a change in quality score as the probe 106 moves towards or farther from a diagnostic-quality view. The portion 606 of the quality score indicator 604 may increase (e.g., a boundary line moves up the quality score indicator 604) as the probe 106 is navigated closer to a diagnostic-quality view. Conversely, as the probe is navigated further from a diagnostic quality view, a boundary line of the portion 606 moves down the quality score indicator 604. In some embodiments, the quality score indicator 604 may include a numerical value indicative of the quality score. The quality score indicator 604 may also include a threshold 608. The threshold 608 may indicate a quality score that has a minimum value that is to be achieved for the corresponding image to be considered a diagnostic quality image.

For example, an image having a quality score of 90 or greater may be considered a diagnostic quality image that can be captured and used for clinical purposes (e.g., diagnoses). As such, the threshold 608 may be a line corresponding to a quality score of 90. As the probe 106 moves nearer a position where a diagnostic quality image is obtained, the portion 606 of the quality score indicator 604 increases towards the threshold 608. For example, when the image achieves a quality score of 90, a boundary line of the portion 606 may be equal to (e.g., overlay upon) the threshold 608.

In various embodiments, the image 610 may be updated in real time as the AI circuit 122 adjusts imaging or acquisition parameters and the quality score changes. For example, as the quality score increases, the AI circuit 122 may increase a frame rate or other imaging parameter, thereby causing the image 610 to increase in quality (e.g., resolution). Further, in some embodiments, the image 610 may decrease in quality. For example, the sonographer may incorrectly move the probe such that the position of the probe 106 is further from a diagnostic view. In another example, the sonographer may capture an image of a first anatomical structure and may move the probe to a different part of the body to image a different anatomical structure. Thus, the Ai circuit 122 may determine that the probe 106 is not near a diagnostic-quality view for the second structure, and may decrease the quality score. The imaging parameters may then be adjusted (e.g., reduced), thereby reducing a quality of the image 610.

Referring now to FIG. 6B an illustration of a guidance system 650 for positioning an ultrasound transducer to corresponding to the illustration of FIG. 6A is shown, according to an example embodiment. The guidance system 650 may be displayed on the same user interface as the ultrasound image 600 (e.g., on the display device 132).

The guidance system 650 includes an animation 652, an image 654, and written guidance 656. The animation 652 may include an animated representation of the tissue to be imaged (shown in FIG. 6B as an animated torso) and an animated probe. The animation 652 may provide a representation to a user (e.g., sonographer) so that the user can mimic the probe placement and movement shown in the animation 652 on a patient. For example, as shown, the animation 652 shows a probe positioned on the left side of a patient's chest. The sonographer may view the animation and similarly place the probe 106 on the left chest of the patient being imaged. The animation 652 may also show movement of the probe (e.g., similar to the probe shown in the guidance instructions 602 of FIG. 6A).

The guidance system 650 further includes an image 654. The image 654 may be the same as or similar to the image 610 of FIG. 6A. That is, the image 654 may be an image representing the anatomy captured by the probe 106. As the probe 106 is moved, the image 654 may change. Further, as the quality score changes, the quality of the image 654 may change. As shown in FIG. 6B, written guidance 656 may be overlaid on the image 654. The written guidance 656 may include instructions for where or how to position the probe. For example, as shown in FIG. 6B, the written guidance states, “Perpendicular parasternal,” “Left sternal edge,” and “3^{rd}-4^{th} intercostal space.” This may indicate where the probe 106 should be positioned relative to the anatomy being imaged. The written guidance 656 may differ from the guidance instructions 602 in that the guidance instructions 602 describe how to move or position the probe, and the written guidance 656 describes where to position the probe.

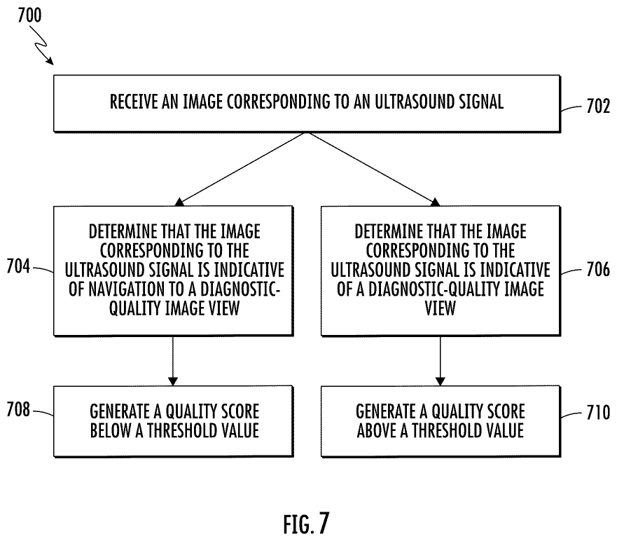

Referring now to FIG. 7, a method 700 for navigating an ultrasound probe 106 is shown, according to an example embodiment.

At process 702, an image corresponding to an ultrasound signal is received. The image may be received by, for example, an artificial intelligence model (e.g., the AI circuit 122).

At process 704, the AI circuit 122 determines that the image corresponding to the ultrasound signal is indicative of navigation to a diagnostic-quality image view. For example, based on the guidance instructions generated for the sonographer by the AI circuit 122, the AI circuit 122 may determine that the probe 106 is not positioned such that a diagnostic view is achieved.

At process 706, the AI circuit 122 determines that the image corresponding to the ultrasound signal is indicative of a diagnostic-quality image view. For example, based on the guidance instructions generated for the sonographer by the AI circuit 122, the AI circuit 122 may determine that the probe 106 is positioned such that a diagnostic view is achieved.

At process 708, responsive to the determination at process 704 that the image corresponding to the ultrasound signal is indicative of navigation to the diagnostic-quality image view, the AI circuit 122 generates a quality score below a threshold value. For example, when the probe 106 has not yet been positioned to capture a view that can be clinically used, the quality score may reflect as such. In such an implementation, the portion 606 of the quality score indicator 604 of FIG. 6A may be below the threshold 608. As will be described herein, upon generation of a quality score below a threshold value, indicating that the probe is not positioned to obtain a diagnostic-quality view of the anatomy, the AI circuit 122 may adjust one or more imaging or acquisition parameters so that a quality of the image (e.g., a sharpness, contrast, beam-space resolution, etc.) corresponding to the ultrasound signal is decreased.

At process 710, responsive to the determination at process 706 that the image corresponding to the ultrasound signal is indicative of the diagnostic-quality image view, the AI circuit 122 generates a quality score above a threshold value. For example, when the probe 106 is positioned to capture a view that can be clinically used, the quality score may reflect as such. In such an implementation, the portion 606 of the quality score indicator 604 of FIG. 6A may be at or above the threshold 608. As will be described herein, upon generation of a quality score at or above the threshold value, the AI circuit 122 may adjust one or more imaging or acquisition parameters so that a quality of the image corresponding to the ultrasound signal is increased to allow capture of the image. In various implementations, the generated image may be captured even when the quality score is below a threshold value (e.g., the probe is not positioned to capture a diagnostic quality image). However, the image may have a lower quality (e.g., resolution, etc.) relative to an image captured after adjustment of the imaging parameters by the AI system 122 when the probe is properly positioned.

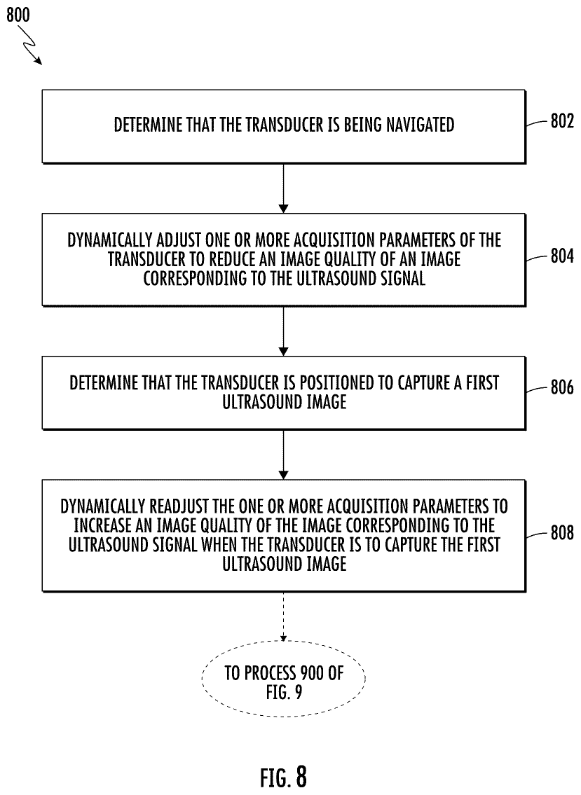

Referring now to FIG. 8, a method 800 for optimizing an ultrasound image quality using artificial intelligence is shown, according to an example embodiment.

At process 802, the AI circuit 122 determines that a transducer (e.g., a transducer of the probe 106) is being navigated. The AI circuit 122 may determine that the transducer is being navigated into a position to capture the first ultrasound image. In some embodiments, at process 802, the AI circuit 122 may not determine that the transducer is being navigated but instead may determine that the transducer is not in a position to obtain a diagnostic-quality image. The AI circuit 122 may make this determination based on one or more guidance instructions generated by the machine learning model. For example, the AI circuit 122 may generate guidance instructions instructing a sonographer how to move or position the probe 106. The AI circuit 122 may determine that the transducer is being navigated by determining that a navigation step is being provided to the sonographer via the guidance system. In some embodiments, process 802 is optional. For example, the AI circuit 122 may not specifically determine that the transducer is being navigated, Instead, the method 800 may begin at process 804 where the AI circuit 122 generates a first quality score so that images of low diagnostic quality can be mapped to low-power acquisition parameters, and images of high diagnostic quality can be mapped to high-power acquisition parameters.

In some embodiments, the method 800 further includes generating, by a machine learning model (e.g., the AI circuit 122), a quality score. The quality score may be indicative of a diagnostic quality of a position of the transducer (e.g., probe). That is, the quality score may indicate how close the image corresponding to the ultrasound signal is to being a diagnostic-quality image. The image corresponding to the ultrasound signal increases as the quality score increases. As such, the AI circuit 122 may determine that the transducer is being navigated based on the quality score being below a threshold value. For example, the AI circuit 122 may determine that the probe 106 is being navigated, and the AI circuit 122 may generate a quality score below a threshold value because the probe 106 is being navigated and is not at a view that is clinically useful.

At process 804, the AI circuit 122 dynamically adjusts one or more acquisition parameters of the transducer to reduce the image quality of an image corresponding to the ultrasound signal. The one or more acquisition parameters may include at least one of a transmission rate, an acquisition rate, a frame rate, or an ultrasound image resolution. For example, because the AI circuit 122 has determined that the probe 106 is being navigated (e.g., using the guidance system), the AI circuit 122 can reduce an image quality of the image because the sonographer or other user is not yet capturing a diagnostic-quality image or otherwise analyzing the image. Only the AI circuit 122 may be analyzing the image, and the AI circuit 122 is capable of adequately analyzing the image when the image quality is less than an image quality needed by the sonographer (e.g., a human) to adequately analyze the image.

At process 806, the AI circuit 122 determines that the transducer is positioned to capture a first ultrasound image. The AI circuit 122 may make this determination by determining that the quality score is above a threshold value. For example, the AI circuit 122 may identify that the guidance instructions have cause the probe to be positioned to capture a diagnostic-quality view. The AI circuit 122 may then generate a quality score for the image that is at or above a threshold value. The first ultrasound image may be captured only when the quality score is at or above the threshold value (e.g., the first ultrasound image may be captured based on determining that the quality score is at or above a threshold value).

At process 808, the AI circuit 122 dynamically readjusts the one or more acquisition parameters. The one or more acquisition parameters may be adjusted to increase the image quality of the image corresponding to the ultrasound signal when the transducer is to capture the first ultrasound image. That is, in some embodiments, the AI circuit 122 may increase the image quality after the probe 106 has been positioned to capture the first ultrasound image but prior to the first ultrasound image actually being captured (e.g., prior to the first ultrasound image being stored). The machine learning model (e.g., the AI circuit 122) dynamically readjusts the one or more acquisition parameters to increase the image quality of the image corresponding to the ultrasound signal based on the determination at process 806 that the quality score is above the threshold value.

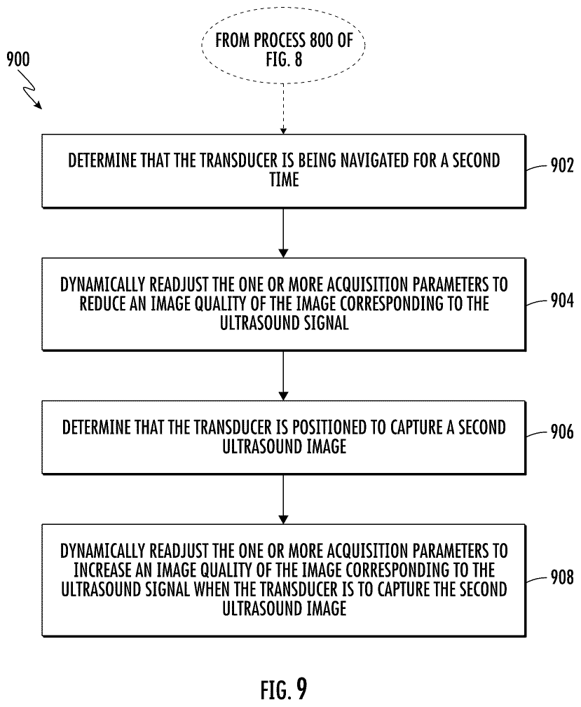

After process 808 has been completed, in some embodiments, the method 800 may continue to a method 900, which will be described herein with respect to FIG. 9.

Referring now to FIG. 9, a method 900 for re-optimizing an ultrasound image quality using artificial intelligence is shown, according to an example embodiment. As stated above, the method 900 may occur responsive to process 808 of FIG. 8 occurring.

At process 902, the AI circuit 122 determines that the transducer is being navigated for a second time. For example, after the transducer has been positioned to capture the first ultrasound image at process 806, the transducer may be moved (e.g., to capture a second anatomical structure, etc.). In some embodiments, the machine learning model (e.g., the AI circuit 122) may decrease the quality score below the threshold value responsive to determining that the transducer is being navigated for the second time. For example, because the probe 106 is no longer positioned to capture a diagnostic-quality image, the quality score may decrease.

At process 904, the AI circuit 122 dynamically readjusts one or more acquisition parameters to reduce the image quality of the image corresponding to the ultrasound signal. The AI circuit 122 may readjust one or more acquisition parameters responsive to the determination at process 902 that the transducer is being navigated for a second time. For example, at process 808, the acquisition parameters may be adjusted to increase the image quality, and at process 904, the acquisition parameters may be adjusted to decrease the image quality because the transducer is being navigated.

At process 906, the AI circuit 122 determines that the transducer is positioned to capture a second ultrasound image. In some embodiments, the method 900 therefore includes increasing, by the machine learning model (e.g., the AI circuit 122), the quality score above the threshold value responsive to determining, at process 906, that the transducer is positioned to capture the second ultrasound image.

At process 908, the AI circuit 122 dynamically readjusts the one or more acquisition parameters to increase an image quality of the image corresponding to the ultrasound signal when the transducer is to capture the second ultrasound image.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be understood that no claim element herein is to be construed under the provisions of 35 U.S.C. §112(f), unless the element is expressly recited using the phrase “means for.”

As utilized herein, terms of degree such as “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to any precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that terms such as “exemplary,” “example,” and similar terms, as used herein to describe various embodiments, are intended to indicate that such embodiments are possible examples, representations, or illustrations of possible embodiments, and such terms are not intended to connote that such embodiments are necessarily extraordinary or superlative examples.

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any element on its own or any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the drawings. It should be noted that the orientation of various elements may differ according to other exemplary embodiments, and that such variations are intended to be encompassed by the present disclosure.

As used herein, terms such as “engine” or “circuit” may include hardware and machine-readable media storing instructions thereon for configuring the hardware to execute the functions described herein. The engine or circuit may be embodied as one or more circuitry components including, but not limited to, processing circuitry, network interfaces, peripheral devices, input devices, output devices, sensors, etc. In some embodiments, the engine or circuit may take the form of one or more analog circuits, electronic circuits (e.g., integrated circuits (IC), discrete circuits, system on a chip (SOCs) circuits, etc.), telecommunication circuits, hybrid circuits, and any other type of circuit. In this regard, the engine or circuit may include any type of component for accomplishing or facilitating achievement of the operations described herein. For example, an engine or circuit as described herein may include one or more transistors, logic gates (e.g., NAND, AND, NOR, OR, XOR, NOT, XNOR, etc.), resistors, multiplexers, registers, capacitors, inductors, diodes, wiring, and so on).

An engine or circuit may be embodied as one or more processing circuits comprising one or more processors communicatively coupled to one or more memory or memory devices. In this regard, the one or more processors may execute instructions stored in the memory or may execute instructions otherwise accessible to the one or more processors. The one or more processors may be constructed in a manner sufficient to perform at least the operations described herein. In some embodiments, the one or more processors may be shared by multiple engines or circuits (e.g., engine A and engine B, or circuit A and circuit B, may comprise or otherwise share the same processor which, in some example embodiments, may execute instructions stored, or otherwise accessed, via different areas of memory).

Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be provided as one or more suitable processors, application specific integrated circuits (ASICs), field programmable gate arrays (FPGAs), digital signal processors (DSPs), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud based processor). Alternatively or additionally, the one or more processors may be internal and/or local to the apparatus. In this regard, a given engine or circuit or components thereof may be disposed locally (e.g., as part of a local server, a local computing system, etc.) or remotely (e.g., as part of a remote server such as a cloud based server). To that end, engines or circuits as described herein may include components that are distributed across one or more locations.

An example system for providing the overall system or portions of the embodiments described herein might include one or more computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data which cause a general purpose computer, special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

Although the drawings may show and the description may describe a specific order and composition of method steps, the order of such steps may differ from what is depicted and described. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions, and arrangement of the embodiments without departing from the scope of the present disclosure as expressed in the appended claims.

What is claimed is:

1. An ultrasound imaging system comprising:
a transducer configured to transmit and receive an ultrasound signal;
a matching layer configured to have an acoustic impedance match between a tissue to be imaged and a material of the transducer;
a damping block configured to absorb ultrasound energy; and
a processing circuit comprising a processor coupled to a memory device storing instructions thereon that, when executed, cause the processing circuit to perform operations comprising:
determining, by a machine learning model, that the transducer is being navigated;
dynamically adjusting, by the machine learning model, one or more acquisition parameters of the transducer to reduce an image quality of an image corresponding to the ultrasound signal;
determining, by the machine learning model, that the transducer is positioned to capture a first ultrasound image; and
dynamically readjusting, by the machine learning model, the one or more acquisition parameters to increase an image quality of the image corresponding to the ultrasound signal when the transducer is positioned to capture the first ultrasound image.

2. The ultrasound imaging system of claim 1, wherein the operations comprise determining that the transducer is being navigated into a position to capture the first ultrasound image based on one or more guidance instructions generated by the machine learning model.

3. The ultrasound imaging system of claim 1, wherein the one or more acquisition parameters comprise at least one of a transmission rate, an acquisition rate, a frame rate, or an ultrasound image resolution.

4. The ultrasound imaging system of claim 1, wherein the operations further comprise:
generating, by the machine learning model, a quality score indicative of a quality of the image corresponding to the ultrasound signal, wherein the quality of the image corresponding to the ultrasound signal increases as the quality score increases.

5. The ultrasound imaging system of claim 4, wherein the operations further comprise:
determining that the quality score is above a threshold value; and
capturing the first ultrasound image based on determining that the quality score is above the threshold value.

6. The ultrasound imaging system of claim 5, wherein the machine learning model dynamically readjusts the one or more acquisition parameters to increase the image quality of the image corresponding to the ultrasound signal based on the determination that the quality score is above the threshold value.

7. The ultrasound imaging system of claim 6, wherein dynamically readjusting the one or more acquisition parameters comprises dynamically readjusting the one or more acquisition parameters for a first time, and wherein the operations further comprise:
determining, by the machine learning model, subsequent to dynamically readjusting the one or more acquisition parameters for the first time, that the transducer is being navigated for a second time;
dynamically readjusting, by the machine learning model, for a second time, the one or more acquisition parameters to reduce an image quality of the image corresponding to the ultrasound signal;
determining, by the machine learning model, that the transducer is positioned to capture a second ultrasound image; and
dynamically readjusting, by the machine learning model, for a third time, the one or more acquisition parameters to increase an image quality of the image corresponding to the ultrasound signal when the transducer is positioned to capture the second ultrasound image.

8. The ultrasound imaging system of claim 7, wherein the operations further comprise:
decreasing, by the machine learning model, the quality score below the threshold value subsequent to determining that the transducer is being navigated for the second time; and
increasing, by the machine learning model, the quality score above the threshold value subsequent to determining that the transducer is positioned to capture the second ultrasound image.

9. The ultrasound imaging system of claim 1, wherein the one or more acquisition parameters of the transducer are adjusted to reduce the image quality of an image corresponding to the ultrasound signal to at least one of: reduce power consumption of the transducer, reduce a temperature of the transducer, or increase a battery life of a probe, the probe comprising the transducer.

10. A medical imaging system comprising:
a processing circuit having a processor coupled to a memory device storing instructions thereon that, when executed, cause the processing circuit to perform operations comprising:
determining, by a machine learning model, that a transducer of the medical imaging system is being navigated, wherein the transducer is configured to transmit and receive an ultrasound signal;
dynamically adjusting, by the machine learning model, one or more acquisition parameters of the transducer to reduce an image quality of an image corresponding to the ultrasound signal;
determining, by the machine learning model, that the transducer is positioned to capture a first ultrasound image; and
dynamically readjusting, by the machine learning model, the one or more acquisition parameters to increase an image quality of the image corresponding to the ultrasound signal when the transducer is positioned to capture the first ultrasound image.

11. The medical imaging system of claim 10, wherein the operations comprise determining that the transducer is being navigated into a position to capture the first ultrasound image based on one or more guidance instructions generated by the machine learning model.

12. The medical imaging system of claim 10, wherein the one or more acquisition parameters comprise at least one of a transmission rate, an acquisition rate, a frame rate, or an ultrasound image resolution.

13. The medical imaging system of claim 10, wherein the operations further comprise:
generating, by the machine learning model, a quality score indicative of a quality of the image corresponding to the ultrasound signal, wherein the image corresponding to the ultrasound signal increases as the quality score increases.

14. The medical imaging system of claim 13, wherein the operations further comprise:
determining that the quality score is above a threshold value; and
capturing the first ultrasound image based on determining that the quality score is above the threshold value.

15. The medical imaging system of claim 14, wherein the machine learning model dynamically readjusts the one or more acquisition parameters to increase the image quality of the image corresponding to the ultrasound signal based on the determination that the quality score is above the threshold value.

16. The medical imaging system of claim 15, wherein dynamically readjusting the one or more acquisition parameters comprises dynamically readjusting the one or more acquisition parameters for a first time, and wherein the operations further comprise:
determining, by the machine learning model, subsequent to dynamically readjusting the one or more acquisition parameters for the first time, that the transducer is being navigated for a second time;
dynamically readjusting, by the machine learning model, for a second time, the one or more acquisition parameters to reduce an image quality of the image corresponding to the ultrasound signal;
determining, by the machine learning model, that the transducer is positioned to capture a second ultrasound image; and
dynamically readjusting, by the machine learning model, for a third time, the one or more acquisition parameters to increase an image quality of the image corresponding to the ultrasound signal when the transducer is positioned to capture the second ultrasound image.

17. The medical imaging system of claim 16, wherein the operations further comprise:
decreasing, by the machine learning model, the quality score below the threshold value subsequent to determining that the transducer is being navigated for the second time; and
increasing, by the machine learning model, the quality score above the threshold value subsequent to determining that the transducer is positioned to capture the second ultrasound image.

18. A method comprising:
determining, by a machine learning model of a processing circuit of an ultrasound imaging system, that a transducer of the ultrasound imaging system is being navigated, wherein the transducer is configured to transmit and receive an ultrasound signal;
dynamically adjusting, by the machine learning model of the processing circuit, one or more acquisition parameters of the transducer to reduce an image quality of an image corresponding to the ultrasound signal;
determining, by the machine learning model of the processing circuit, that the transducer is positioned to capture a first ultrasound image; and
dynamically readjusting, by the machine learning model of the processing circuit, the one or more acquisition parameters to increase an image quality of the image corresponding to the ultrasound signal when the transducer is positioned to capture the first ultrasound image.

19. The method of claim 18, wherein dynamically readjusting the one or more acquisition parameters comprises dynamically readjusting the one or more acquisition parameters for a first time, the method further comprising:
determining, by the machine learning model of the processing circuit, subsequent to dynamically readjusting the one or more acquisition parameters for the first time, that the transducer is being navigated for a second time;
dynamically readjusting, by the machine learning model of the processing circuit, for a second time, the one or more acquisition parameters to reduce an image quality of the image corresponding to the ultrasound signal;
determining, by the machine learning model of the processing circuit, that the transducer is positioned to capture a second ultrasound image; and
dynamically readjusting, by the machine learning model of the processing circuit, for a third time, the one or more acquisition parameters to increase an image quality of the image corresponding to the ultrasound signal when the transducer is positioned to capture the second ultrasound image.

20. The method of claim 19, further comprising:
decreasing, by the machine learning model of the processing circuit, a quality score below a threshold value subsequent to determining that the transducer is being navigated for the second time; and
increasing, by the machine learning model of the processing circuit, the quality score above the threshold value subsequent to determining that the transducer is positioned to capture the second ultrasound image.
